(19) United States
(12) Patent Application Publication
(10) Pub. No.: US 2021/0128049 A1
(43) Pub. Date: May 6, 2021

(54) PRONUNCIATION FUNCTION EVALUATION SYSTEM BASED ON ARRAY HIGH-DENSITY SURFACE ELECTROMYOGRAPHY

(71) Applicant: SHENZHEN INSTITUTES OF ADVANCED TECHNOLOGY, Shenzhen (CN)

(72) Inventors: Shixiong CHEN, Shenzhen (CN); Mingxing ZHU, Shenzhen (CN); Guanglin LI, Shenzhen (CN); Zijian YANG, Shenzhen (CN); Jiashuo ZHUANG, Shenzhen (CN); Xiaochen WANG, Shenzhen (CN); Xin WANG, Shenzhen (CN)

(21) Appl. No.: 16/927,948

(22) Filed: Jul. 13, 2020

Related U.S. Application Data
(63) Continuation-in-part of application No. PCT/CN2019/130813, filed on Dec. 31, 2019.

(30) Foreign Application Priority Data
Mar. 25, 2019 (CN) 201910228434.X

(51) Int. Cl. A61B 5/394; A61B 5/296; A61B 5/397; A61B 5/00; G16H 50/50

(52) U.S. Cl. CPC A61B 5/394; A61B 5/296; A61B 5/397; A61B 5/4803; A61B 5/6822; A61B 5/0004; G16H 50/50; A61B 5/6814; A61B 5/7242

(57) ABSTRACT

A pronunciation function evaluation system based on array high-density surface electromyography, this system includes a host computer and a slave computer, the slave computer is configured to obtain faciocervical electromyography signal through electromyography electrode arrays in a pronunciation process and to transmit the faciocervical electromyography signals to the host computer; the host computer is configured to analyze a physiological relevance between faciocervical array high-density electromyography signal features change and pronunciation function in the pronunciation process, to establish a three-dimensional dynamic energy distribution diagram of faciocervical muscular movement in the pronunciation process, to obtain dynamic visual temporal and spatial characteristic of articulatory muscles related to pronunciation, to extract electromyography features, to establish a faciocervical electromyography feature distribution standard database with normal pronunciation function, and to analyze a dysfunction condition and a damage degree of the pronunciation muscle group using a template matching and differentiation analysis algorithm.

### CROSS-REFERENCE TO RELATED APPLICATION

The present application is a Continuation-in-Part of PCT Patent Application Serial No. PCT/CN2019/130813, filed on Dec. 31, 2019, which claims priority to Chinese patent application Serial No. 201910228434.X, filed on Mar. 25, 2019, the entire contents of which are incorporated herein by reference in their entireties.

### TECHNICAL FIELD

The present disclosure relates to the technical field of pronunciation function evaluation and diagnosis, and particularly to a pronunciation function evaluation system based on array high-density surface electromyography.

### BACKGROUND

Pronunciation is an important activity via which nature sounds are produced for people to perform information communication. The speech generated from the pronunciation process contains information such as people's feelings and emotions and is a significant manifestation of social communication capability. Speech communication is a basic function of the survival of human society; whether sound may be sent out in a lossless manner is the key to express people's own emotions. Pronunciation is a complex process controlled by multiple articulatory muscles located on the facial and neck regions. During a pronunciation process, airflow from the lung passes through the throat, muscle is contracted, cartilage is enabled to be activated, and the vocal cord is driven to move simultaneously so that the vocal cord is relaxed or tensioned, the glottis is opened or closed with relaxing or tensioning of the vocal cord, and the sound is generated accordingly. The loudness of the sound is depended on the tightness of the vocal cords. The pitch of the sound was affected by the length, tension, quality, and position of the vocal cords when vibrating. Whether vocal organs can work normally and orderly is dependent on the coordination of the central nervous system and the corresponding muscles associated with pronunciation, abnormity occurs in any working link of the vocal organs would lead to pronunciation dysfunction.

Pronunciation dysfunction is a common disease and is prone to become sequela or complication of the related disease. An investigation showed that about 75% to 90% of patients having Parkinson's disease had pronunciation dysfunction, either, the occurrence rate of pronunciation dysfunction for patients having cerebral apoplexy was between 30% and 40%; moreover, in a severe case, 15% of the patients having cerebral apoplexy had pronunciation dysfunction for a long time. These symptoms impacted the patient's daily communication capabilities seriously and imposed a heavy burden on the family and society. In another aspect, with the progress and the development of the era, people's life became more diversified, and the number of people who were engaged in the occupations such as speech, singing, and education is increasing, these people also required high-quality sound for working to improve their professional levels. Research showed that nearly 31% of sales personnel, 44% of athletic coaches, and 58% of teachers had different levels of pronunciation dysfunction that impacted people's work in related art seriously. The relevant report indicated that there are about 28 million jobs closely related to the voice in the United States of America, about 7.2% of labors lose their jobs due to dysarthria each year, where the proportion of teachers who lose their jobs due to pronunciation dysfunction is up to 20%, the economic loss caused by unemployment which was due to pronunciation dysfunction is up to 2.5 billion US dollars. According to the incomplete statistics, there were about 1.37 million patients in China who are unable to speak normally due to loss of pronunciation functionality currently. Therefore, timely and accurate evaluation and detection of pronunciation function have great significance for social development and medical progress.

In the previous studies, the evaluation of pronunciation functions was mostly based on speech signals. However, the speech signals had a much higher requirement for the environment. Once a test environment is noisy, it is difficult to transmit the voice signal accurately. The speech signals were characterized by the acoustic parameters. The vocal cords did not vibrate during the pronunciation of voiceless sound, voice feature is unobvious, which causes a decline of evaluation for pronunciation function. Pronunciation is a complex neuromuscular activity, so the physiological features during the pronunciation process may not be assessed by using the speech signals.

Pronunciation is a complex neuromuscular activity controlled by the coordination of a large group of small facial and neck muscles that span a relatively large area. Speed, force, range, direction, and coordination of movement of the relevant muscle groups have an influence on the pronunciation function, an occurrence of dysfunction in any one of the pronunciation muscles may cause pronunciation dysfunction, and the damage degree of the sound is closely related to the damage degree of nerve-muscle.

The surface electromyography signal is an electrical signal generated during muscle contraction. There is a strong correlation between the electromyography signal and the function of the muscles, which may reflect the activity level of the corresponding nerve-muscle to different degrees. The surface electromyography signal is a bio-electrical signal recorded by surface electrodes arranged on the skin surfaces when the muscles contract and generate excitement. Since the surface electromyography technique has the advantages of non-invasive, being easy to operate, being low in the cost, being capable of providing quantitative and qualitative analysis, etc., it has been widely used in medical detection and biofeedback treatment. The previous investigations of pronunciation were based on a single or a few channels of electrodes. However, the pronunciation process requires a mass of articulatory muscles located on the facial and neck regions, and most of the muscles were in small size. Therefore, a few electrodes could lead to the loss of useful information related to pronunciation functions, which would affect pronunciation evaluation. Thus, it is difficult to use a single or a few electrodes to evaluate the pronunciation function. There is a lack of approaches to early and accurately recognize the individual's pronunciation function features clinically, which leads to the lack of individualization and refinement of the existing pronunciation rehabilitation training strategy, resulting in poor rehabilitation.

### Technical Problems

In view of this, the embodiments of the present disclosure provide a pronunciation function evaluation system based on array high-density surface electromyography, which aims at solving a problem in the previous investigation that a few electrodes cannot provide enough electrophysiological information of the entire pronunciation process, which lead to the unreliable evaluation of the pronunciation function as well as the lack of individualization and refinement of the existing voice rehabilitation training strategy.

### Technical Solution

The pronunciation function evaluation system based on array high-density surface electromyography provided by the embodiments of the present disclosure may include:

a slave computer configured to obtain faciocervical electromyography signals through surface electrode arrays in a pronunciation process, and to transmit the faciocervical electromyography signals to a host computer; and

the host computer being configured to analyze a physiological relevance between faciocervical array high-density surface electromyography signal features change and pronunciation function in the pronunciation process, to establish a three-dimensional dynamic energy distribution diagram of faciocervical muscular movement in the pronunciation process, to obtain dynamic visual temporal and spatial characteristic of the articulatory muscles related to pronunciation, to extract electromyography features, to establish a faciocervical electromyography feature distribution standard database with normal pronunciation function, and to analyze a dysfunction condition and a damage degree of the pronunciation muscle group using a template matching and differentiation analysis algorithm.

Further, the host computer is configured to:

receive the electromyography signals transmitted from the slave computer, filter power interference and baseline drift through a preset filter, and filter interference noise in the electromyography signal through a preset optimization algorithm;

extract time-domain and frequency-domain features from the electromyography signals, extract the root mean square, as a time-domain feature, from the electromyography signals with a series of analysis windows, make the strength of the muscles associated with pronunciation to be corresponding to color so as to form faciocervical three-dimensional dynamic energy distribution maps, and obtain the dynamic visual temporal and spatial characteristic of the articulatory muscles related to pronunciation; and

extract the electromyography features, establish the faciocervical electromyography feature distribution standard database with normal pronunciation function, and analyze the dysfunction condition and the damage degree of the pronunciation muscle group using the template matching and differentiation analysis algorithm.

Further, the host computer is configured to:

modularize a calculation algorithm of the electromyographic features in the pronunciation process, package the calculation algorithm into a separate function control function, and display the dysfunction condition and the damage degree of the pronunciation muscle group on a GUI in real-time.

Furthermore, the optimization algorithm includes an independent component analysis algorithm, a principal component analysis algorithm, and a template matching algorithm.

Furthermore, the surface electromyography features include the time-domain features, frequency-domain features, faciocervical energy distribution ratio, and muscle synergies.

Furthermore, the time-domain features include average electromyography, integrated electromyography, root mean square, zero-crossing rate, and electromyography variance;

the frequency-domain features include power spectral density, median frequency, average power frequency, peak frequency, average power, and frequency ratio of the surface electromyography signals;

the faciocervical energy distribution ratio include relative area of energy, relative width of energy, and energy gradient of the energy maps;

the muscle synergies include the amount structure of synergies as well as the coefficient of synergies.

Furthermore, the slave computer includes:

electromyography electrode arrays configured to obtain the faciocervical electromyography signals during the pronunciation process; and

an electromyography acquisition circuit configured to transmit the faciocervical electromyography signals to the host computer.

Furthermore, the electromyography electrode arrays include two pieces of surface electromyography electrodes in an 4×5 array on the face and two pieces of surface electromyography electrodes in an 8×5 array on the neck, respectively.

Furthermore, the electromyography acquisition circuit includes a microcontroller, a right leg drive, analog-to-digital converter, an independent synchronization clock, a signal filtering preamplifier, and a low noise power supply.

Furthermore, the electromyography acquisition circuit is configured to feedback the electromyography signal to human body through the right leg driving and perform common-mode rejection on the electromyography signals, to filter and amplify the electromyography signals through the signal filtering preamplifier and transmit the electromyography signals to the analog-to-digital converter, to realize synchronous real-time acquisition of the electromyography signals under the control of the independent synchronization clock, to transmit the electromyography signals to the microcontroller, and to further transmit the electromyography signals to the host computer through Wi-Fi.

### Advantageous Effects

Compared with the prior technique, the technological effects of the embodiments of the present disclosure are as follows: the pronunciation function evaluation system based on array high-density surface electromyography includes a slave computer and a host computer, the slave computer is configured to obtain faciocervical electromyography signals through the electromyography electrode arrays in the pronunciation process, and to transmit the faciocervical electromyography signals to the host computer; the host computer is configured to analyze the physiological relevance between the faciocervical array high-density electromyography signals features change and the pronunciation function in the pronunciation process, to establish three-dimensional dynamic energy distribution maps of faciocervical muscular activities in the pronunciation process, to obtain temporal and spatial characteristic of the articulatory muscles related to pronunciation, to extract electromyography features, to establish a faciocervical electromyography features distribution standard database with normal pronunciation function, and to use a template matching and differentiation analysis algorithm to analyze a dysfunction condition and a damage degree of the pronunciation muscle group. To overcome the limitation of speech signals, the surface electromyography signals are used to evaluate the electrophysiological function of pronunciation in the present disclosure. Due to the fact that the electromyography signals have the advantages of low environmental requirement, high stability, stronger anti-interference capability, etc., there is a stronger physiological correlation between the surface electromyography signals and the pronunciation function, the faciocervical surface electromyography signals in the pronunciation process are collected for analysis, so that the features of the physiological function of pronunciation activity may be effectively evaluated. Furthermore, in the embodiments of the present disclosure, the electromyography signals are collected through the electromyography electrode arrays, so that the electrophysiological features of the pronunciation muscle groups in the pronunciation process are analyzed more completely and objectively, and performing quantitative or qualitative analysis on the neural function and the muscles is finally realized by collecting the electromyography signals during muscle activities in the pronunciation process, the evaluation of pronunciation function is enabled to be visualized and refined, real-time, objective and accurate evaluation of pronunciation function is realized.

### BRIEF DESCRIPTION OF THE DRAWINGS

In order to explain the embodiments of the present disclosure more clearly, a brief introduction regarding the accompanying drawings that need to be used for describing the embodiments of the present disclosure or the prior art is given below; it is apparent that the accompanying drawings described as follows are merely some embodiments of the present disclosure, the person of ordinary skill in the art may also acquire other drawings according to the current drawings on the premise of paying no creative labor.

FIG. 1 is a structural block diagram of a pronunciation function evaluation system based on array high-density surface electromyography provided by an embodiment of the present disclosure; and

FIG. 2 is a structural block diagram of a slave computer provided by an embodiment of the present disclosure.

### DESCRIPTION OF THE EMBODIMENTS

In order to make the purpose, technical features, and advantages of the present disclosure be more obvious and more understandable, technical solutions in this embodiment of the present disclosure will be described clearly and comprehensively with reference to accompanying drawings in this embodiment. And it is obvious that this embodiment described below is merely part of this embodiment of the present disclosure, but not the whole of this embodiment. Based on this embodiment in the present disclosure, some other embodiments, which are obtained by one of ordinary skill in the art at the premise of paying no creative labor, are all included in the protection scope of the present disclosure.

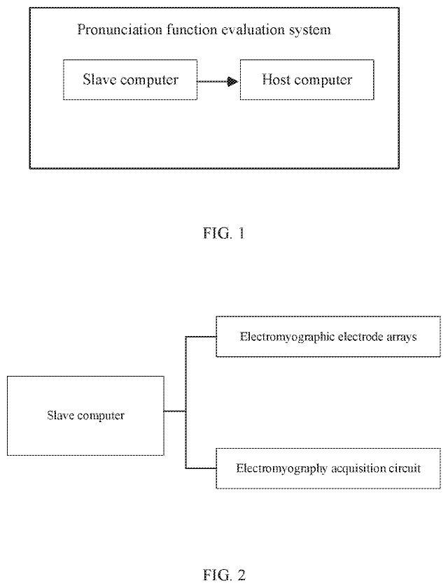

FIG. 1 illustrates a structural block diagram of the pronunciation function evaluation system based on the array high-density surface electromyography. For the convenience of description, the part associated with this embodiment is merely illustrated.

Referring to FIG. 1, a pronunciation function evaluation system based on the array high-density surface electromyography provided in this embodiment of the present disclosure may include a slave computer and a host computer.

The slave computer is configured to obtain faciocervical electromyography signals in a pronunciation process through electromyography electrode arrays, and to transmit the faciocervical electromyography signals to the host computer.

The host computer is configured to analyze physiological relevance between faciocervical array high-density electromyography signal features change and pronunciation function in the pronunciation process, to establish three-dimensional dynamic energy distribution maps of faciocervical muscular movement in the pronunciation process, to obtain dynamic visual temporal and spatial characteristic of articulatory muscles related to pronunciation, to extract the electromyography features, to establish a faciocervical electromyography feature distribution standard database with normal pronunciation function, and to analyze a dysfunction condition and a damage degree of the pronunciation muscle group using a template matching and differentiation analysis algorithm.

As shown in FIG. 2, the slave computer may include electromyography electrode arrays and an electromyography acquisition circuit.

The electromyography electrode arrays are configured to obtain faciocervical electromyography signals in the pronunciation process.

In a particular implementation of this embodiment, the electromyography electrode arrays may include two pieces of surface electromyography electrodes (20 channels) in an 4×5 array on the face and surface electromyography electrodes (40 channels) in the array of 8×5 on the neck. The number of electrodes may be increased according to the requirement of the user, the number of electrodes is as many as 120 channels. The surface electromyography electrode is a round or square gold-plated electrode, different diameters of electrodes may be set according to the requirement. All electrodes are attached on a flexible substrate equidistantly with smaller interval so that an array electrode sheet which may be bent and is closely attached to the skin is formed, multi-channel and high-density electromyography signals on the surface of the user's skin may be obtained in real-time. Since electromyography electrodes of up to 120 channels may be used to obtain more comprehensive electromyography signals, when the electrode array is adopted, the spacing of the electrodes is small, and more detailed information may be obtained; the shape of the electrode may be changed according to a bending degree of the skin when the flexible electrode is adopted, the flexible electrodes may be better attached with the skin, and the obtained electromyography signals are more stable and reliable.

The electromyography acquisition circuit is configured to transmit the faciocervical electromyography signals to the host computer.

In one specific implementation of this embodiment, the electromyography acquisition circuit may include main parts such as microcontroller, right leg drive, analog-to-digital converter, independent synchronous clock, signal filtering preamplifier, low-noise power supply, etc., where the microcontroller is integrated with Wi-Fi (Wireless Fidelity) function. The electromyography acquisition circuit is specifically configured to feedback the electromyography signals to human body through the right leg drive and perform signal common-mode rejection on the electromyography signal, the electromyography signals are filtered and amplified through the signal filtering preamplifier and are transmitted to the analog-to-digital converter, synchronous and real-time collection of the multi-channel electromyography signals is realized under the control of the independent synchronous clock, the multi-channel electromyography signals are transmitted to the microcontroller and are sent to the host computer through Wi-Fi. Since the wireless transmission is more convenient and is simpler in wearing as compared to the traditional wired electrode, and the influence caused by winding of wires of wired electrodes is reduced. Data loss is avoided during Wi-Fi transmission, so that data integrity is ensured. The multi-channel electromyography signals are simultaneously transmitted wirelessly, so that a defect that the traditional wireless electrodes have less channels and incomplete information is remedied.

Preferably, the host computer is configured to receive the electromyography signals transmitted by the slave computer, to filter power interference and baseline drift through a preset filter, and to filter interference noise in the electromyography signals through a preset optimization algorithm. In one aspect, the amount of information of original data is saved to the maximum; in another aspect, signal quality is improved, and reliable data is provided for further functional feature analysis. Where the filter may include a high-pass standard filter and/or a low-pass standard filter and the like. the optimization algorithm may include an ICA (Independent Component Analysis) algorithm, a PCA (Principal Component Analysis) algorithm and/or a template matching algorithm, etc., and the interference noise includes artifacts and/or electrocardio, and the like.

Preferably, the host computer is configured to extract time-domain and frequency-domain features from the electromyography signals, to extract root mean square, as a time-domain feature, from the electromyography signals with a series of analysis windows, to make the strength of the muscles associated with pronunciation to be corresponding to color by performing a software algorithm, the faciocervical three-dimensional dynamic energy distribution maps are formed, the dynamic visual space-time information of the articulatory muscles related to pronunciation is obtained, and temporal and spatial characteristic of the multi-channel electromyography collected by the faciocervical pronunciation muscle group are visualized. Meanwhile, frequency-domain analysis is performed on the electromyography to extract the electromyography frequency spectrum distribution maps and the time-frequency distribution maps, the distribution feature of electromyography frequency-domain in the pronunciation process is visualized. In this way, the dynamic change information of the pronunciation muscle group in the pronunciation process is provided, the dynamic movement condition under cooperative work of the pronunciation muscle group is obtained intuitively and in real time, and a problem that dynamic information of activities of related pronunciation muscle groups in the pronunciation process may not be obtained intuitively and in real-time at the present stage is solved.

Preferably, the host computer is configured to extract features from the preprocessed multi-channel electromyography signals, to extract electromyography features, to establish a faciocervical electromyography feature distribution standard database with normal pronunciation function, and to analyze the dysfunction condition and the damage degree of the pronunciation muscle group by collecting faciocervical multi-channel array high-density surface electromyography signal of the person who is to be detected and performing template matching and differentiation analysis on the faciocervical array high-density surface electromyography signals and database features with normal pronunciation function. In this way, the pronunciation function is comprehensively analyzed from multiple aspects, objective quantitative evaluation is achieved, and the reliability of function analysis of the pronunciation muscle group is ensured. The faciocervical electromyography feature distribution standard database with normal pronunciation function is established, the deficiency of electromyography features with normal pronunciation function is remedied, the faciocervical array high-density surface electromyography information of the person who is to be detected is collected, the template matching and differentiation analysis is performed on the faciocervical array high-density surface electromyography information and the database feature with normal pronunciation function, the dysfunction condition and the damage degree of the pronunciation muscle group are analyzed, a damage degree is evaluated for the pronunciation dysfunction, and precision evaluation of the pronunciation function is realized.

The electromyography features may include time-domain features, frequency-domain features, faciocervical energy distribution ratio, and muscle synergies, etc. The time-domain features may include AEMG (Average Electromyography), an iEMG (integral Electromyography), RMS (Root Mean Square) value, a ZCR (Zero-Crossing Rate), and electromyography variance, etc., the frequency-domain features may include PSD (Power Spectral Density), MF (Median Frequency), meanpower frequency, peak frequency, mean power, and frequency ratio, etc., the faciocervical energy distribution ratio may include relative area of energy, relative width of energy and/or energy gradient, etc., the muscle synergies may include the amount structure of the synergies as well as the coefficient of synergies. In addition, there are also time-frequency method, spatial method, chaotic and fractal method that may provide features.

Preferably, the host computer is provided with a GUI (Graphic User Interface) and is configured to modularize the calculation algorithm of the electromyographic features in the pronunciation process (which may include the calculation algorithms of features of electromyographic waveforms, faciocervical energy distribution, electromyography spectrum distribution, time-domain features, frequency-domain features, faciocervical energy distribution ratio, and muscle synergistic distribution), to package the calculation algorithm into a separate function control function, and to display the dysfunction condition and the damage degree of the pronunciation muscle group on the GUI in real-time.

The foregoing refers to acquisition and analysis of the electromyography information of the faciocervical pronunciation muscle group; in addition, muscles associated with pronunciation function such as abdomen also contain certain pronunciation activity information, and may also be used as the electromyography information source of this embodiment to perform pronunciation function evaluation.

As stated above, the pronunciation function evaluation system based on array high-density surface electromyography provided by the embodiment of the present disclosure includes the slave computer and the host computer, where the slave computer is configured to obtain the faciocervical electromyography signals through the electromyography electrode arrays in the pronunciation process, and to transmit the faciocervical electromyography signals to the host computer; the host computer is configured to analyze the physiological relevance between the faciocervical array high-density electromyography signal features change and the pronunciation function in the pronunciation process, to establish the three-dimensional dynamic energy distribution maps of faciocervical muscular movement in the pronunciation process, to obtain the dynamic visual temporal and spatial characteristic of the articulatory muscles related to pronunciation, to extract the electromyography features, to establish the faciocervical electromyography feature distribution standard database with normal pronunciation function, and to analyze the dysfunction condition and the damage degree of the pronunciation muscle group using the template matching and differentiation analysis algorithm. To overcome the limitation of speech signals, the surface electromyography signal is used to evaluate the electrophysiological function of pronunciation in the present disclosure. Due to the fact that the electromyography signals have the advantages of low environmental requirement, high stability, stronger anti-interference capability, etc., there is a stronger physiological correlation between the surface electromyography signals and the pronunciation function, the faciocervical surface electromyography signal in the pronunciation process is collected for analysis, so that the features of physiological function of pronunciation activity may be effectively evaluated. Furthermore, in the embodiments of the present disclosure, the electromyography signals are collected through the electromyography electrode array, so that the electrophysiological features of the pronunciation muscle groups in the pronunciation process are analyzed more completely and objectively, and performing quantitative or qualitative analysis on the neural function and the muscles is finally realized by collecting the electromyography signals during muscle activities in the pronunciation process, the evaluation of pronunciation function is enabled to be visualized and refined, real-time, objective and accurate evaluation of pronunciation function is realized.

It may be clearly understood by the person of ordinary skill in the art that, for the convenience of description and conciseness, dividing of the aforesaid various functional units, functional modules is described exemplarily merely, in actual application, the aforesaid functions may be assigned to different functional systems and functional modules to be accomplished so as to accomplish the whole or a part of functionalities described above. The various functional systems, modules in the embodiments may be integrated into a processing unit, or each of the units exists independently and physically, or two or more than two of the units are integrated into a single unit. The aforesaid integrated unit may by either actualized in the form of hardware or in the form of software functional units. In addition, specific names of the various functional units and modules are only used to distinguish from each other conveniently, rather than being intended to limit the protection scope of the present disclosure.

The embodiments described above are only intended to explain but not to limit the technical solutions of the present disclosure. Although the present disclosure has been explained in detail with reference to the above-described embodiments, it should be understood by the ordinary skilled one in the art that, the technical solutions described in each of the embodiments mentioned above can still be amended, or some technical features in the technical solutions can be replaced equivalently; these amendments or equivalent replacements, which doesn't cause the essence of corresponding technical solution to be broken away from the spirit and the scope of the technical solution in various embodiments of the present disclosure, should all be included in the protection scope of the present disclosure.

What is claimed is:

1. A pronunciation function evaluation system based on array high-density surface electromyography, comprising:
a slave computer configured to obtain faciocervical electromyography signals through surface electrodes arrays in a pronunciation process, and to transmit the faciocervical electromyography signals to a host computer; and
a host computer configured to analyze a physiological relevance between faciocervical array high-density electromyography signal features change and pronunciation function in the pronunciation process, to establish a three-dimensional dynamic energy distribution diagram of faciocervical muscular movement in the pronunciation process, to obtain dynamic visual temporal and spatial characteristic of articulatory muscles related to pronunciation, to extract electromyography features, to establish a faciocervical electromyography feature distribution standard database with normal pronunciation function, and to analyze a dysfunction condition and a damage degree of the pronunciation muscle group using a template matching and differentiation analysis algorithm.

2. The system according to claim 1, wherein the host computer is configured to:
receive the electromyography signal transmitted from the slave computer, to filter power interference and baseline drift through a preset filter, and to filter interference noise in the electromyography signals through a preset optimization algorithm;
extract time-domain and frequency-domain features from the electromyography signals, extract root mean square, as a time-domain feature, from the electromyographic signals with a series of analysis windows, make the strength of the muscles associated with pronunciation to be corresponding to color so as to form faciocervical three-dimensional dynamic energy distribution maps, and obtain dynamic visual temporal and spatial characteristic of the articulatory muscles related to pronunciation; and
extract the electromyography features, establish the faciocervical electromyography feature distribution standard database with normal pronunciation function, and analyze the dysfunction condition and the damage degree of the pronunciation muscle group using the template matching and differentiation analysis algorithm.

3. The system according to claim 2, wherein the host computer is configured to modularize a calculation algorithm of the electromyography features in the pronunciation process, to package the calculation algorithm into a separate function control function, and to display the dysfunction condition and the damage degree of the pronunciation muscle group on a GUI in real-time.

4. The system according to claim 2, wherein the optimization algorithm comprises an independent component analysis algorithm, a principal component analysis algorithm, and a template matching algorithm.

5. The system according to claim 2, wherein the surface electromyography features comprises time-domain features, frequency-domain features, faciocervical energy distribution ratio, and muscle synergies.

6. The system according to claim 5, wherein the time-domain features comprise average electromyography value, integrated electromyography, root mean square, zero-crossing rate, and electromyography variance;
the frequency-domain features comprise power spectral density, median frequency, average power frequency, peak frequency, average power, and frequency ratio of the surface electromyographic signals;
the faciocervical energy distribution ratio comprises relative area of energy, relative width of energy, and energy gradient of energy maps;
the muscle synergies includes the amount structure of synergies as well as the coefficient of synergies.

7. The system according to claim 1, wherein the slave computer comprises:
electromyography electrode arrays configured to obtain the faciocervical electromyography signals in the pronunciation process; and
an electromyography acquisition circuit configured to transmit the faciocervical electromyography signals to the host computer.

8. The system according to claim 7, wherein the electromyography electrode arrays comprise two pieces of surface electromyography electrodes in an 4×5 array on the face and two pieces of surface electromyography electrodes in an 8×5 array on the neck, respectively.

9. The system according to claim 7, wherein the electromyography acquisition circuit comprises a microcontroller, a right leg drive, an analog-to-digital converter, an independent synchronization clock, a signal filtering preamplifier, and a low noise power supply.

10. The system according to claim 9, wherein the electromyography acquisition circuit is configured to feedback the electromyography signals to human body through the right leg driving and perform common-mode rejection on the electromyography signals, to filter and amplify the electromyography signals through the signal filtering preamplifier and transmit the electromyography signals to the analog-to-digital converter, to realize synchronous real-time acquisition of the electromyography signals under the control of the independent synchronization clock, to transmit the electromyography signals to the microcontroller, and to further transmit the electromyography signals to the host computer through Wi-Fi.
